(19) United States
(12) Patent Application Publication
(10) Pub. No.: US 2024/0294537 A1
(43) Pub. Date: Sep. 5, 2024

(54) SOLID STATE FORMS OF 4-[[4-(4-CHLOROANILINO)FURO[2,3-d]PYRIDAZIN-7-yl]OXYMETHYL]-N-METHYLPYRIDINE-2-CARBOXAMIDE AND SALT THEREOF

(71) Applicant: TEVA PHARMACEUTICALS INTERNATIONAL GMBH, Jona (CH)

(72) Inventors: Parven Kumar Luthra, Noida (IN); Anantha Rajmohan Muthusamy, Sivakasi (IN); Meenakshi Sundaram Somasundaram, Virudu Nagar Dt (IN); Rahul Kumar Reddy Putikum, Mahabubnagar (IN)

(21) Appl. No.: 18/567,414

(22) PCT Filed: Jun. 29, 2022
(86) PCT No.: PCT/US2022/035413

§ 371 Date: Dec. 6, 2023

(30) Foreign Application Priority Data
Jul. 7, 2021 (IN) 202111030504
Jul. 20, 2021 (IN) 202111032580
Sep. 9, 2021 (IN) 202111040848
Apr. 4, 2022 (IN) 202211020232

(51) Int. Cl. C07D 491/048; A61K 31/5025

(52) U.S. Cl. CPC C07D 491/048; A61K 31/5025

(57) ABSTRACT

The present disclosure relates to solid state forms of Telatinib and Telatinib mesylate, processes for their preparation, and pharmaceutical compositions thereof (I).

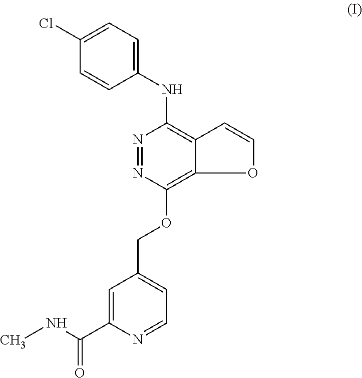 [SMILES: CNC(=O)c1cc(COc2nnc(Nc3ccc(Cl)cc3)c3ccoc23)ccn1]

### TECHNICAL FIELD

The present disclosure relates to solid state forms of Telatinib and Telatinib mesylate, processes for their preparation, and pharmaceutical compositions thereof.

### BACKGROUND

Telatinib has the chemical name 4-[[4-(4-chloroanilino)furo[2,3-d]pyridazin-7-yl]oxymethyl]-N-methylpyridine-2-carboxamide, and the code name BAY 57-9352. Telatinib has the following chemical structure:

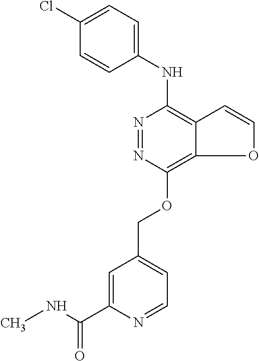 [SMILES: CNC(=O)c1cc(COc2nnc(Nc3ccc(Cl)cc3)c3ccoc23)ccn1]

Telatinib is an orally active small molecule that blocks tumour angiogenesis by inhibiting VEGFR-2, VEGFR-3, PDGFR-β, and c-kit tyrosine kinase activity, indicated for the treatment of cancer, specifically for gastric cancer. Telatinib is also under investigation for the treatment of gastroesophageal junction cancer, hepatocellular carcinoma, advanced solid tumors, and pseudomyogenic hemangioendothelioma. Telatinib is disclosed in U.S. Pat. No. 6,689,883. International Publication No. WO 2007/118602 discloses a process for preparing Telatinib and its salts including mesylate salt.

Solid state form of Telatinib mesylate, Mod. I, is disclosed in International Publication No. WO 2018/188495 and solid state forms of Telatinib are described in CN 112830932.

Polymorphism, the occurrence of different crystal forms, is a property of some molecules and molecular complexes. A single compound, like Telatinib or salt thereof, may give rise to a variety of polymorphs having distinct crystal structures and physical properties like melting point, thermal behaviors (e.g., measured by thermogravimetric analysis—“TGA”, or differential scanning calorimetry—“DSC”), powder X-ray diffraction (XRPD) pattern, infrared absorption fingerprint, Raman absorption fingerprint, and solid state (¹³C—) NMR spectrum. One or more of these techniques may be used to distinguish different polymorphic forms of a compound.

Different salts and solid state forms (including solvated forms and co-crystals) of an active pharmaceutical ingredient may possess different properties. Such variations in the properties of different salts and solid state forms, co-crystals profile, or improving stability (polymorph as well as chemical stability) and shelf and solvates may provide a basis for improving formulation, for example, by facilitating better processing or handling characteristics, improving the dissolution-life. These variations in the properties of different salts and solid state forms may also provide improvements to the final dosage form, for instance, if they serve to improve bioavailability. Different salts and solid state forms, co-crystals and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts, solid state forms, co-crystals and solvates of a pharmaceutical product can provide materials having desirable processing properties, such as ease of handling, ease of processing, storage stability, and ease of purification, or as desirable intermediate crystal forms that facilitate conversion to other salts or polymorphic forms. New salts, polymorphic forms, co-crystals and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life. For at least these reasons, there is a need for additional solid state forms (including solvated or hydrated forms) of Telatinib. Furthermore, there is a need for additional solid state forms of Telatinib mesylate which offer superior properties without altering the pharmacological properties.

### SUMMARY

The present disclosure relates to solid state forms of Telatinib and Telatinib mesylate, processes for preparation, and to pharmaceutical compositions thereof.

In particular, the present disclosure provides crystalline forms of Telatinib designated as Forms T1-T6 (defined herein) and salts of Telatinib mesylate, including: Telatinib mono mesylate Forms TM2 and TM5 and Telatinib dimesylate Forms TM1, TM3 and TM4 (defined herein).

The present disclosure also provides uses of the solid state forms of Telatinib and Telatinib mesylate or combination thereof for preparing other solid state forms of Telatinib and Telatinib mesylate.

In another embodiment, the present disclosure encompasses the herein described solid state forms of Telatinib and Telatinib mesylate for use in the preparation of pharmaceutical compositions and/or formulations, in some embodiments for the treatment of cancer, or in some embodiments, for the treatment of gastric cancer, gastroesophageal junction cancer, hepatocellular carcinoma, advanced solid tumors, and pseudomyogenic hemangioendothelioma, and particularly gastric cancer.

Another embodiment of the present disclosure encompasses the use of the herein described solid state forms of Telatinib and Telatinib mesylate for the preparation of pharmaceutical compositions and/or formulations.

The present disclosure further provides pharmaceutical compositions including solid state forms of Telatinib and Telatinib mesylate.

In yet another embodiment, the present disclosure encompasses pharmaceutical formulations including combinations of the solid state forms of Telatinib and Telatinib mesylate and at least one pharmaceutically acceptable excipient. The pharmaceutical composition or formulation includes oral dosage forms, e.g., a tablet or capsule.

The present disclosure encompasses processes to prepare said pharmaceutical formulations of solid state forms of Telatinib and Telatinib mesylate, including combining any one or a combination of the solid state forms of Telatinib and Telatinib mesylate according to the present disclosure with at least one pharmaceutically acceptable excipient. The process can optionally include adding additional active agent(s).

The solid state forms of Telatinib and Telatinib mesylate, as well as the pharmaceutical compositions or formulations of solid state forms of Telatinib and Telatinib mesylate according to the present disclosure, can be used as medicaments, in embodiments for the treatment of cancer, or in embodiments for the treatment of gastric cancer. The solid state forms of Telatinib and telatinib mesylate, pharmaceutical compositions and formulations thereof, as described in any aspect or embodiment of the present invention, can be used for the treatment of gastric cancer, gastroesophageal junction cancer, hepatocellular carcinoma, advanced solid tumors, and pseudomyogenic hemangioendothelioma, and particularly gastric cancer.

The present disclosure also provides methods of treating cancer, gastroesophageal junction cancer, hepatocellular carcinoma, advanced solid tumors, and pseudomyogenic hemangioendothelioma. and particularly for the treatment of gastric cancer, by administering a therapeutically effective amount of any one or a combination of the solid state forms of Telatinib and Telatinib mesylate according to the present disclosure, or at least one of the above pharmaceutical compositions or formulations, to a subject suffering from the cancer, or to a subject suffering from gastric cancer, or otherwise in need of treatment.

The present disclosure also provides uses of solid state forms of Telatinib and Telatinib mesylate of the present disclosure, or at least one of the above pharmaceutical compositions or formulations, for the manufacture of a medicament for treating cancer, or for the manufacture of a medicament for treating gastric cancer, gastroesophageal junction cancer, hepatocellular carcinoma, advanced solid tumors, and pseudomyogenic hemangioendothelioma, and particularly gastric cancer.

### BRIEF DESCRIPTION OF THE FIGURES

FIG. 1 shows an X-ray powder diffraction pattern (XRPD) of Telatinib Form T1.

FIG. 2 shows an X-ray powder diffraction pattern (XRPD) of Telatinib Form T2.

FIG. 3 shows an X-ray powder diffraction pattern (XRPD) of Telatinib Form T3.

FIG. 4 shows an X-ray powder diffraction pattern (XRPD) of Telatinib Form T4.

FIG. 5 shows an X-ray powder diffraction pattern (XRPD) of micronized Telatinib mesylate Mod. I, as described in PCT Patent Application Publication No. WO 2018/188495 (FIG. 2).

FIG. 6 shows an X-ray powder diffraction pattern (XRPD) of Telatinib dimesylate Form TM1.

FIG. 7 shows an X-ray powder diffraction pattern (XRPD) of Telatinib mono mesylate Form TM2.

FIG. 8 shows an XRPD pattern of Telatinib dimesylate Form TM3.

FIG. 9 shows an XRPD pattern of Telatinib Form T5.

FIG. 10 shows an XRPD pattern of Telatinib Form T6.

FIG. 11 shows an XRPD pattern of Telatinib dimesylate Form TM4.

FIG. 12 shows an XRPD pattern of Telatinib mono mesylate Form TM5.

### DETAILED DESCRIPTION

The present disclosure relates to solid state forms of Telatinib and Telatinib mesylate, including Telatinib mono mesylate and Telatinib dimesylate, to processes for preparation thereof, and to pharmaceutical compositions including these solid state forms or combinations thereof.

The solid state forms of Telatinib and Telatinib mesylate according to the present disclosure may have advantageous properties including at least one of: chemical or polymorphic purity, flowability, solubility, dissolution rate, bioavailability, morphology or crystal habit, stability such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Telatinib and Telatinib mesylate referred to herein as being characterized by graphical data “as depicted in” a Figure will thus be understood to include any crystal forms of the Telatinib and Telatinib mesylate, characterized with the graphical data having such small variations, as are well known to the skilled person, in comparison with the Figure.

A solid state form (or polymorph) may be referred to herein as polymorphically pure or substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or about 0% of any other forms of the subject compound as measured, for example, by XRPD. Thus, solid state forms of Telatinib and/or Telatinib mesylate described herein as substantially free of any other solid state forms would be understood to contain greater than about 80% (w/w), greater than about 90% (w/w), greater than about 95% (w/w), greater than about 98% (w/w), greater than about 99% (w/w), or about 100% (w/w) of the subject solid state forms of Telatinib and/or Telatinib mesylate. Accordingly, in some embodiments of the disclosure, the described solid state forms of Telatinib and/or Telatinib mesylate may contain from about 1% to about 20% (w/w), from about 5% to about 20% (w/w), or from about 5% to about 10% (w/w) of one or more other solid state forms of Telatinib and/or Telatinib mesylate.

As used herein, Telatinib mesylate refers to Telatinib mesylate salt, including Telatinib mono mesylate, wherein the molar ratio between Telatinib and methanesulfonic acid is 1:1, and Telatinib dimesylate, wherein the molar ratio between Telatinib and methanesulfonic acid is 1:2.

The modifier “about” should be considered as disclosing the range defined by the absolute values of the two endpoints. For example, the expression “from about 2 to about 4” also discloses the range “from 2 to 4.” When used to modify a single number, the term “about” may refer to plus or minus 10% of the indicated number and includes the indicated number. For example, “about 10%” may indicate a range of 9% to 11%, and “about 1” means from 0.9-1.1.

As used herein, unless stated otherwise, XRPD peaks reported herein are preferably measured using CuK_{α} radiation, λ=1.5418 Å.

As used herein, the term “isolated” in reference to solid state forms of Telatinib and Telatinib mesylate are physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C. A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

The term “solvate”, as used herein and unless indicated otherwise, refers to a crystal form that incorporates a solvent in the crystal structure. When the solvent is water, the solvate is often referred to as a “hydrate”. The solvent in a solvate may be present in either a stoichiometric or in a non-stoichiometric amount.

As used herein, and unless stated otherwise, the term “anhydrous” in relation to crystalline forms of Telatinib and Telatinib mesylate, relates to a crystalline form of Telatinib and Telatinib mesylate which does not include any crystalline water (or other solvents) in a defined, stoichiometric amount within the crystal. Moreover, an “anhydrous” form would typically not contain more than 1% (w/w), of either water or organic solvents as measured, for example, by TGA. In embodiments, the crystalline form of Telatinib and Telatinib mesylate contains less than 1% (w/w), less than 0.5% (w/w) and less than 0.2% of water or organic solvents as measured by TGA.

The amount of solvent employed in a chemical process, e.g., a reaction or a crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of about 10 mbar to about 50 mbar.

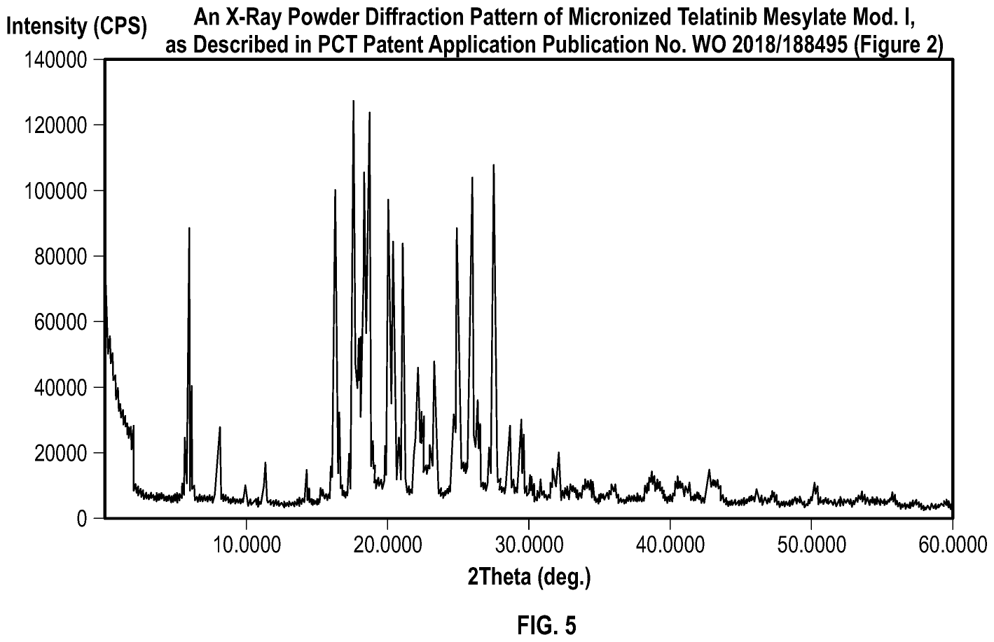

As used herein, the term “micronized Mod. I” is micronized crystalline Telatinib mesylate Mod. I described in PCT Publication No. WO 2018/188495. For example, as defined in WO2 018/188495, micronized Mod. I has an XRPD pattern with characteristic peaks at: 4.01, 7.85, 9.94, 13.04, 19.08, 19.46, 20.10, 21.82, 22.49, 23.76, 24.26, 27.17, 28.52 and 30.48 degrees 2-theta±0.1 degrees 2-theta, micronized Mod. I may also be defined with reference to the XRPD in FIG. 5 herein.

The present disclosure includes solid state forms of Telatinib.

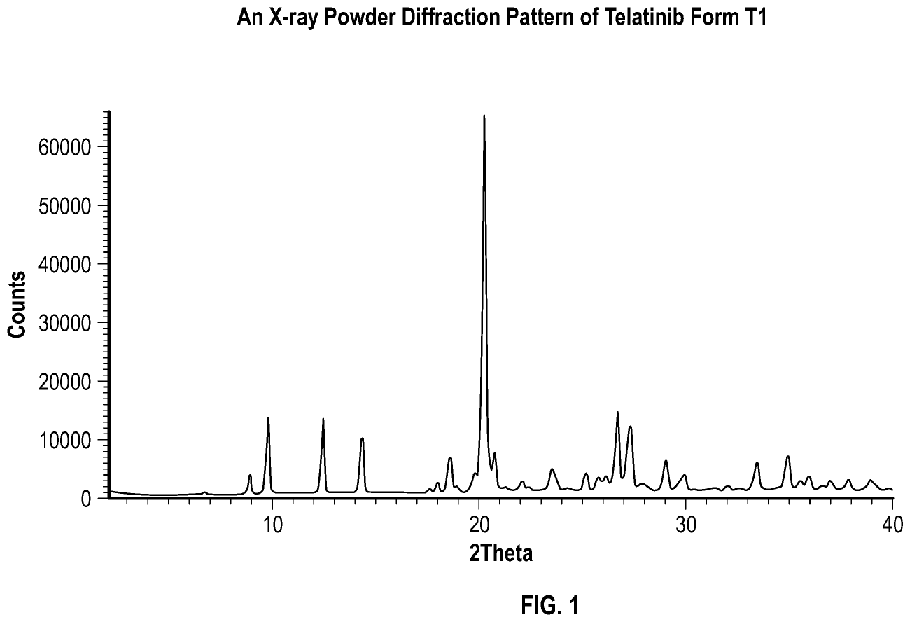

The present disclosure further includes a crystalline form of Telatinib designated as Form T1. Telatinib Form T1 can be characterized by data selected from one or more of the following: an XRPD pattern having peaks at 8.9, 9.8, 20.3, 26.7 and 27.9 degrees 2-theta±0.2 degrees 2-theta; an XRPD pattern as depicted in FIG. 1; or combinations of these data.

Telatinib Form T1 may be further characterized by an XRPD pattern having peaks at 8.9, 9.8, 20.3, 26.7 and 27.9 degrees 2-theta±0.2 degrees 2-theta, and also having one, two, three or four additional peaks at 12.5, 14.4, 18.6 and 29.0 degrees 2-theta±0.2 degrees 2-theta.

Telatinib Form T1 may alternatively be characterized by an XRPD pattern having peaks at 8.9, 9.8, 12.5, 14.4, 18.6, 20.3, 26.7, 27.9 and 29.0 degrees 2-theta±0.2 degrees 2-theta.

Telatinib Form T1 may be characterized by each of the above characteristics alone or by all possible combinations, e.g., by an XRPD pattern having peaks at 8.9, 9.8, 20.3, 26.7 and 27.9 degrees 2-theta±0.2 degrees 2-theta and/or an XRPD pattern as depicted in FIG. 1.

In another embodiment, Telatinib Form T1 may optionally be characterized as a hydrate, preferably as monohydrate. Telatinib Form T1 may contain from about 3 to about 5% of water, preferably about 4.2% of water, for example as measured by Karl Fischer instrument or by TGA (theoretical stoichiometric amount of one water molecule is equal to 4.2%).

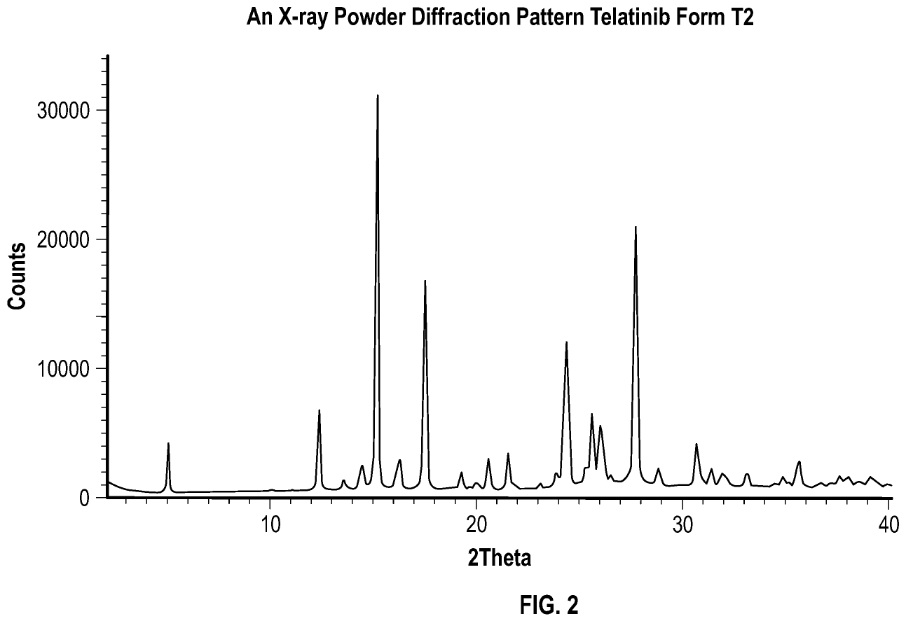

The present disclosure further includes a crystalline form of Telatinib designated as Form T2. Telatinib Form T2 can be characterized by data selected from one or more of the following: an XRPD pattern having peaks at 5.1, 15.2, 16.3, 27.7, 28.8 and 30.7 degrees 2-theta±0.2 degrees 2-theta; an XRPD pattern as depicted in FIG. 2; or combinations of these data.

Telatinib Form T2 may be further characterized by an XRPD pattern having peaks at 5.1, 15.2, 16.3, 27.7, 28.8 and 30.7 degrees 2-theta±0.2 degrees 2-theta, and also having one, two, three or four additional peaks at 12.4, 17.5, 24.4 and 25.6 degrees 2-theta±0.2 degrees 2-theta.

Telatinib Form T2 may alternatively be characterized by an XRPD pattern having peaks at 5.1, 12.4, 15.2, 16.3, 17.5, 24.4, 25.6, 27.7, 28.8 and 30.7 degrees 2-theta±0.2 degrees 2-theta.

Telatinib Form T2 may be characterized by each of the above characteristics alone or by all possible combinations, e.g., by an XRPD pattern having peaks at 5.1, 15.2, 16.3, 27.7, 28.8 and 30.7 degrees 2-theta±0.2 degrees 2-theta and/or an XRPD pattern as depicted in FIG. 2.

In another embodiment Telatinib Form T2 may be characterized as anhydrous form.

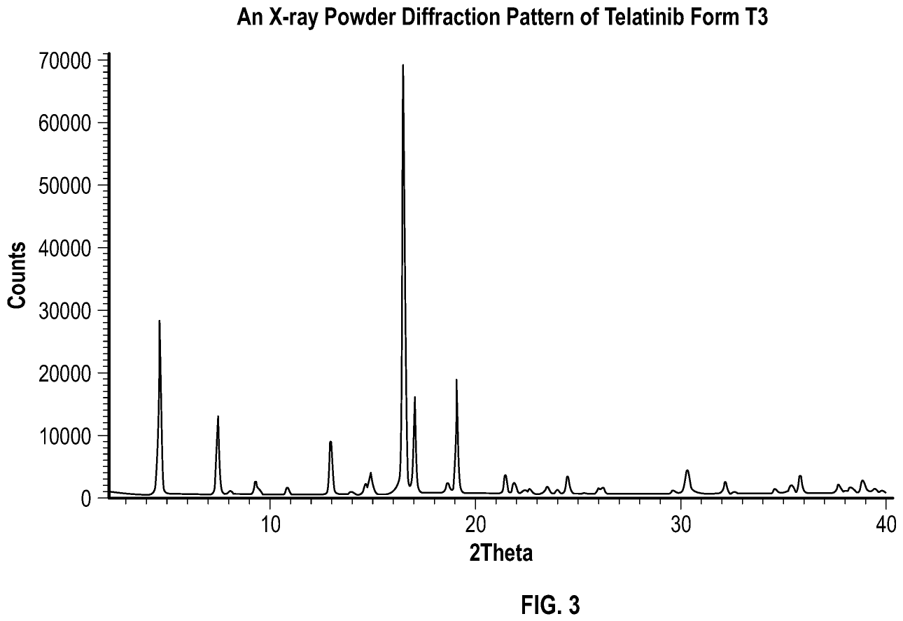

The present disclosure further includes a crystalline form of Telatinib designated as Form T3. Telatinib Form T3 can be characterized by data selected from one or more of the following: an XRPD pattern having peaks at 4.7, 7.5, 9.3, 13.0 and 16.6 degrees 2-theta±0.2 degrees 2-theta; an XRPD pattern as depicted in FIG. 3; or combinations of these data.

Telatinib Form T3 may be further characterized by an XRPD pattern having peaks at 4.7, 7.5, 9.3, 13.0 and 16.6 degrees 2-theta±0.2 degrees 2-theta, and also having one, two, three or four additional peaks at 17.1, 19.1, 21.5 and 22.7 degrees 2-theta±0.2 degrees 2-theta.

Telatinib Form T3 may alternatively be characterized by an XRPD pattern having peaks at 4.7, 7.5, 9.3, 13.0, 16.6, 17.1, 19.1, 21.5 and 22.7 degrees 2-theta±0.2 degrees 2-theta.

Telatinib Form T3 may be characterized by each of the above characteristics alone or by all possible combinations, e.g., by an XRPD pattern having peaks 4.7, 7.5, 9.3, 13.0 and 16.6 degrees 2-theta±0.2 degrees 2-theta and/or an XRPD pattern as depicted in FIG. 3.

In another embodiment Telatinib Form T3 may be characterized as anhydrous form.

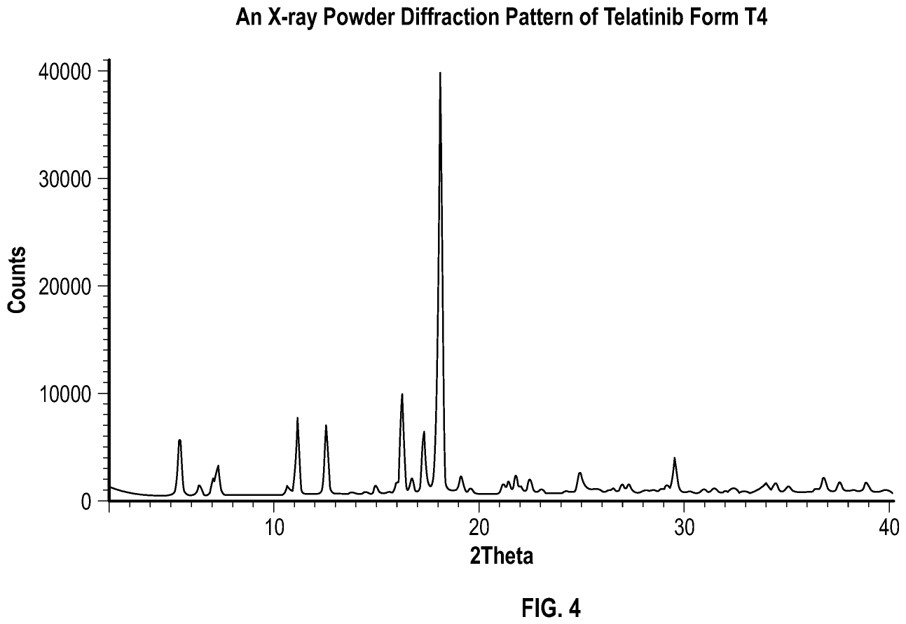

The present disclosure further includes a crystalline form of Telatinib designated as Form T4. Telatinib Form T4 can be characterized by data selected from one or more of the following: an XRPD pattern having peaks at 5.4, 11.2, 17.3, 18.2 and 24.9 degrees 2-theta±0.2 degrees 2-theta; an XRPD pattern as depicted in FIG. 4; or combinations of these data.

Telatinib Form T4 may be further characterized by an XRPD pattern having peaks at 5.4, 11.2, 17.3, 18.2 and 24.9 degrees 2-theta±0.2 degrees 2-theta, and also having one, two, three or four additional peaks at 6.4, 12.6, 16.3 and 29.5 degrees 2-theta±0.2 degrees 2-theta.

Telatinib Form T4 may alternatively be characterized by an XRPD pattern having peaks at 5.4, 6.4, 11.2, 12.6, 16.3, 17.3, 18.2, 24.9 and 29.5 degrees 2-theta±0.2 degrees 2-theta.

Telatinib Form T4 may be characterized by each of the above characteristics alone or by all possible combinations, e.g., by an XRPD pattern having peaks at 5.4, 11.2, 17.3, 18.2 and 24.9 degrees 2-theta±0.2 degrees 2-theta and/or an XRPD pattern as depicted in FIG. 4.

In another embodiment, Telatinib Form T4 may optionally be characterized as ethylene glycol solvate, preferably mono ethylene glycol solvate.

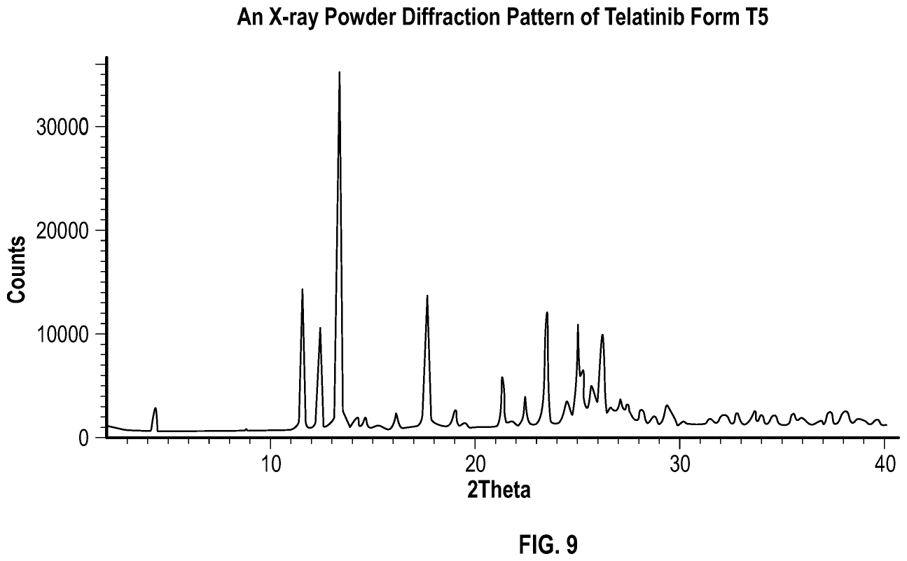

The present disclosure further includes a crystalline form of Telatinib designated as Form T5. Telatinib Form T5 can be characterized by data selected from one or more of the following: an XRPD pattern having peaks at 4.4, 11.7, 13.5, 17.8 and 25.1 degrees 2-theta±0.2 degrees 2-theta; an XRPD pattern as depicted in FIG. 9; or combinations of these data.

Telatinib Form T5 may be further characterized by an XRPD pattern having peaks at 4.4, 11.7, 13.5, 17.8 and 25.1 degrees 2-theta±0.2 degrees 2-theta, and also having one, two, three, four or five additional peaks at 12.5, 16.2, 21.4, 23.5 and 26.2 degrees 2-theta±0.2 degrees 2-theta.

Telatinib Form T5 may alternatively be characterized by an XRPD pattern having peaks at 4.4, 11.7, 12.5, 13.5, 16.2, 17.8, 21.4, 23.5, 25.1 and 26.2 degrees 2-theta±0.2 degrees 2-theta.

Telatinib Form T5 may be characterized by each of the above characteristics alone or by all possible combinations, e.g., by an XRPD pattern having peaks at 4.4, 11.7, 13.5, 17.8 and 25.1 degrees 2-theta±0.2 degrees 2-theta and/or an XRPD pattern as depicted in FIG. 9.

In another embodiment, Telatinib Form T5 may optionally be characterized as 1,4-Dioxane solvate, preferably mono 1,4-Dioxane solvate.

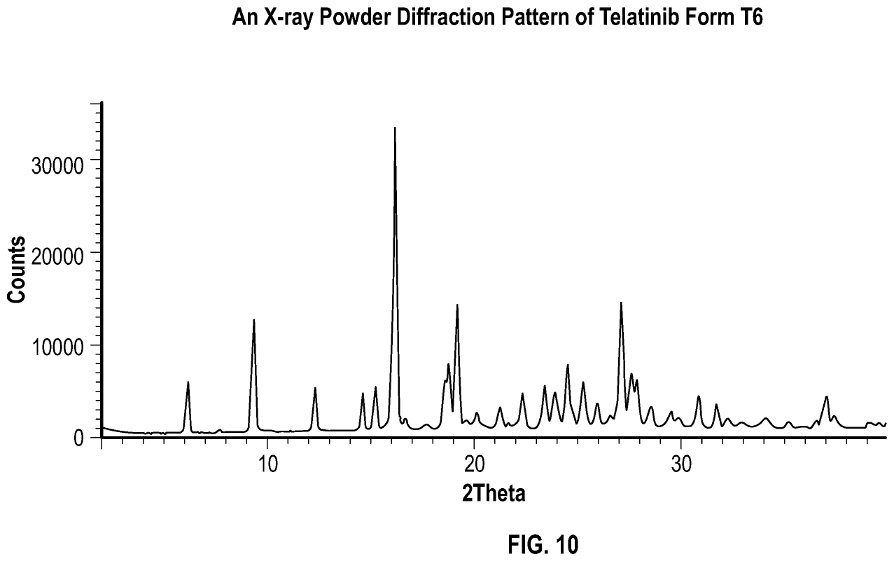

The present disclosure further includes a crystalline form of Telatinib designated as Form T6. Telatinib Form T6 can be characterized by data selected from one or more of the following: an XRPD pattern having peaks at 6.2, 9.4, 12.3, 16.2 and 19.2 degrees 2-theta±0.2 degrees 2-theta; an XRPD pattern as depicted in FIG. 10; or combinations of these data.

Telatinib Form T6 may be further characterized by an XRPD pattern having peaks at 6.2, 9.4, 12.3, 16.2 and 19.2 degrees 2-theta±0.2 degrees 2-theta, and also having one, two, three, four or five additional peaks at 14.6, 22.2, 25.3, 27.2 and 30.9 degrees 2-theta±0.2 degrees 2-theta.

Telatinib Form T6 may alternatively be characterized by an XRPD pattern having peaks at 6.2, 9.4, 12.3, 14.6, 16.2, 19.2, 22.2, 25.3, 27.2 and 30.9 degrees 2-theta±0.2 degrees 2-theta.

Telatinib Form T6 may be characterized by each of the above characteristics alone or by all possible combinations, e.g., by an XRPD pattern having peaks at 6.2, 9.4, 12.3, 16.2 and 19.2 degrees 2-theta±0.2 degrees 2-theta and/or an XRPD pattern as depicted in FIG. 10.

In another embodiment, Telatinib Form T6 may optionally be characterized as anhydrous form.

The present disclosure includes solid state forms of Telatinib mesylate.

In embodiment, the present disclosure includes solid state forms of Telatinib mono mesylate and Telatinib dimesylate.

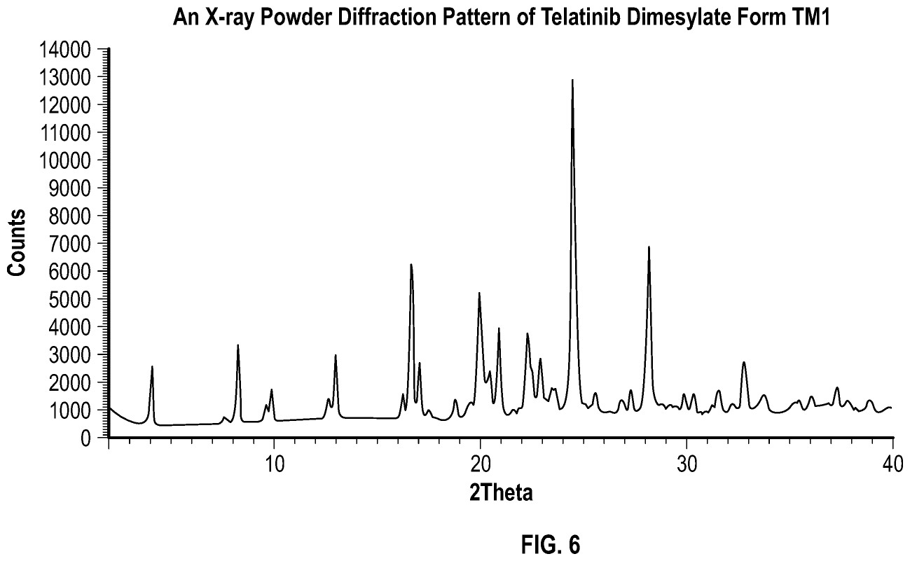

The present disclosure further includes a crystalline form of Telatinib dimesylate designated as Form TM1. Telatinib dimesylate Form TM1 can be characterized by data selected from one or more of the following: an XRPD pattern having peaks at 8.3, 17.0, 20.9, 22.9 and 28.2 degrees 2-theta±0.2 degrees 2-theta.; an XRPD pattern as depicted in FIG. 6; or combinations of these data.

Telatinib dimesylate Form TM1 may be further characterized by an XRPD pattern having peaks at 8.3, 17.0, 20.9, 22.9 and 28.2 degrees 2-theta±0.2 degrees 2-theta, and also having one, two, three or four additional peaks at 16.2, 20.3, 24.5 and 25.6 degrees 2-theta±0.2 degrees 2-theta. Alternatively, Telatinib dimesylate Form TM1 may be characterized by an XRPD pattern having peaks at 8.3, 16.2, 17.0, 20.3, 20.9, 22.9, 24.5, 25.6 and 28.2 degrees 2-theta±0.2 degrees 2-theta.

Telatinib dimesylate Form TM1 may be characterized by an XRPD pattern having peaks at 8.3, 17.0, 20.9, 22.9 and 28.2 degrees 2-theta±0.2 degrees 2-theta and any one, two or three additional peaks at: 9.9, 13.0 and 16.7 degrees 2-theta±0.2 degrees 2-theta, or an XRPD pattern having peaks at: 8.3, 9.9, 13.0, 16.7, 17.0, 20.9, 22.9 and 28.2 degrees 2-theta±0.2 degrees 2-theta.

Telatinib dimesylate Form TM1 may alternatively be characterized by an XRPD pattern having peaks at 8.3, 9.9, 13.0, 16.7, 17.0, 20.9, 22.9 and 28.2 degrees 2-theta±0.2 degrees 2-theta, and also having one, two, three or four additional peaks at 16.2, 20.3, 24.5, and 25.6 degrees 2-theta±0.2 degrees 2-theta. Alternatively, Telatinib dimesylate Form TM1 may be characterized by an XRPD pattern having peaks at 8.3, 9.9, 13.0, 16.2, 16.7, 17.0, 20.3, 20.9, 22.9, 24.5, 25.6 and 28.2 degrees 2-theta±0.2 degrees 2-theta.

Telatinib dimesylate Form TM1 may be characterized by each of the above characteristics alone or by all possible combinations, e.g., by an XRPD pattern having peaks at 8.3, 17.0, 20.9, 22.9 and 28.2 degrees 2-theta±0.2 degrees 2-theta; by an XRPD pattern having peaks at 8.3, 9.9, 13.0, 16.7, 17.0, 20.9, 22.9 and 28.2 degrees 2-theta±0.2 degrees 2-theta and/or an XRPD pattern as depicted in FIG. 6.

In another embodiment, Telatinib dimesylate Form TM1 may optionally be characterized as anhydrous. Thus, Form TM1 as described according to any aspect or embodiment of the present disclosure, may be further characterized as being an anhydrous form.

According to any aspect or embodiment of the present disclosure, Telatinib dimesylate Form TM1 is polymorphically pure. Optionally, Telatinib dimesylate Form TM1 may contain: about 20% or less, about 10% or less, about 5% or less, about 2% or less, about 1% or less, about 0.5% or less, of any other solid state forms of the subject compound as measured, for example, by XRPD.

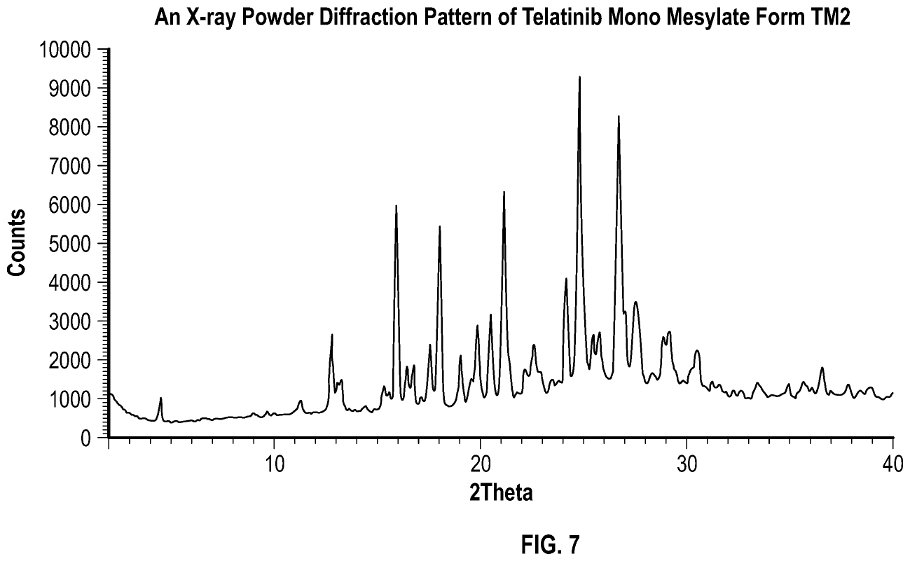

The present disclosure further includes a crystalline form of Telatinib mono mesylate designated as Form TM2. Telatinib mono mesylate Form TM2 can be characterized by data selected from one or more of the following: an XRPD pattern having peaks at 16.0, 16.5, 18.0, 22.6 and 24.9 degrees 2-theta±0.2 degrees 2-theta; an XRPD pattern having peaks at 16.0, 16.5, 18.0, 19.0, 22.6 and 24.9 degrees 2-theta±0.2 degrees 2-theta, an XRPD pattern having peaks at 16.0, 16.5, 18.0, 22.6 and 24.9 degrees 2-theta±0.2 degrees 2-theta and the absence of a peak at 7.9 degrees 2-theta±0.2 degrees 2-theta; an XRPD pattern having peaks at 16.0, 16.5, 18.0, 19.0, 22.6 and 24.9 degrees 2-theta±0.2 degrees 2-theta and the absence of a peak at 7.9 degrees 2-theta±0.2 degrees 2-theta; an XRPD pattern as depicted in FIG. 7; or combinations of these data.

Telatinib mono mesylate form TM2 disclosed herein may be characterized by an XRPD pattern as described in any of the aspects or embodiments described herein, and also having one, two, three or four additional peaks at 4.5, 12.8, 21.2 and 26.8 degrees 2-theta±0.2 degrees 2-theta. For example, Telatinib mono mesylate Form TM2 may be further characterized by an XRPD pattern having peaks at 16.0, 16.5, 18.0, 22.6 and 24.9 degrees 2-theta±0.2 degrees 2-theta, and also having one, two, three or four additional peaks at 4.5, 12.8, 21.2 and 26.8 degrees 2-theta±0.2 degrees 2-theta.

Alternatively, Telatinib mono mesylate Form TM2 according to any aspect or embodiment of the present disclosure may be characterized by an XRPD pattern having peaks at 16.0, 16.5, 18.0, 19.0, 22.6 and 24.9 degrees 2-theta±0.2 degrees 2-theta and also having one, two, three or four additional peaks at 4.5, 12.8, 21.2 and 26.8 degrees 2-theta±0.2 degrees 2-theta, and optionally the absence of peak at 7.9 degrees 2-theta±0.2 degrees 2-theta.

Telatinib mono mesylate Form TM2 may alternatively be characterized by an XRPD pattern having peaks at 4.5, 12.8, 16.0, 16.5, 18.0, 21.2, 22.6, 24.9 and 26.8 degrees 2-theta±0.2 degrees 2-theta.

Telatinib mono mesylate Form TM2 according to any aspect or embodiment of the present disclosure may alternatively be characterized by an XRPD pattern having peaks at 4.5, 12.8, 16.0, 16.5, 18.0, 19.0, 21.2, 22.6, 24.9 and 26.8 degrees 2-theta±0.2 degrees 2-theta and the absence of peak at 7.9 degrees 2-theta±0.2 degrees 2-theta.

Telatinib mono mesylate Form TM2 may be characterized by each of the above characteristics alone or by all possible combinations, e.g., by an XRPD pattern having peaks at 16.0, 16.5, 18.0, 22.6 and 24.9 degrees 2-theta±0.2 degrees 2-theta; an XRPD pattern having peaks at 16.0, 16.5, 18.0, 19.0, 22.6 and 24.9 degrees 2-theta±0.2 degrees 2-theta and the absence of peak at 7.9 degrees 2-theta±0.2 degrees 2-theta; and/or an XRPD pattern as depicted in FIG. 7.

In another embodiment, Telatinib mono mesylate Form TM2 may optionally be characterized as a hydrate. Thus, Telatinib mono mesylate Form TM2 as described in any aspect or embodiment of the present disclosure, may be further characterized as a hydrate. Telatinib mono mesylate Form TM2 according to any aspect or embodiment of the present disclosure may contain from about 1 to about 5% of water, for example as measured by Karl Fischer instrument or by TGA.

According to any aspect or embodiment of the present disclosure, Telatinib mono mesylate Form TM2 is isolated.

According to any aspect or embodiment of the present disclosure, Telatinib mono mesylate Form TM2 is polymorphically pure. Optionally, Telatinib mono mesylate Form TM2 may contain: about 20% or less, about 10% or less, about 5% or less, about 2% or less, about 1% or less, about 0.5% or less, of any other solid state forms of the subject compound as measured, for example, by XRPD.

Telatinib mono mesylate Form TM2 is stable upon exposure to high humidity. Telatinib mono mesylate is stable when exposed to 20-100% RH at 25° C. for 7 days.

The present disclosure provides a process for preparing Telatinib mono mesylate Form TM2, comprising (a) slurrying a mixture of Telatinib mono mesylate and an ether, preferably a C₄-C₈ ether or a C₄-C₆ ether, and more particularly, methyl tert-butyl ether, and (b) optionally isolating the Telatinib mono mesylate Form TM2 from the mixture in step (a). Step (a) may stirring the mixture of Telatinib mono mesylate for a sufficient time to form Telatinib mono mesylate Form TM2. Optionally, the mixture may be stirred at a temperature of about 20° C. to about 30° C., or about 22° C. to about 27° C., or about 25° C. Optionally, the stirring may be for a time period of about 10 minutes to about 24 hours, about 20 minutes to about 12 hours, about 30 minutes to about 6 hours, about 1 hour to about 4 hours, or about 2 hours. Optionally, the Telatinib mono mesylate may be isolated from the mixture in step (a). The isolation may be by any suitable procedure, for example, centrifugation, decantation or filtration, preferably filtration. After isolation, the Telatinib mono mesylate may be dried. The drying may be carried out under vacuum, optionally at a temperature of about 30° C. to about 90° C., about 40° C. to about 80° C., or about 50° C. to about 70° C., or about 60° C. The drying may be for any suitable period of time, for example about 20 minutes to about 4 hours, about 30 minutes to about 2 hours, about 45 minutes to about 1.5 hours, or about 1 hour.

According to any aspect or embodiment of this process, the mixture of Telatinib mono mesylate and ether is a suspension, which may be prepared by a process comprising: (i) suspending Telatinib, preferably Telatinib Form T2, in the ether to form a suspension; and (ii) combining the suspension with methanesulfonic acid. In step (i), the Telatinib may be suspended in the ether at a temperature of about 20° C. to about 30° C., or about 22° C. to about 27° C., or about 25° C. The suspension in step (i) may stirred, optionally for a time period of about 5 minutes to about 4 hours, about 10 minutes to about 2 hours, about 20 minutes to about 1 hour, or about 30 minutes. Optionally, the suspension in step (i) may be stirred for about 30 minutes at 25° C. The ether, preferably methyl tert-butyl ether, may be used in an amount of: about 2 to about 60 ml, about 5 to about 50 ml, about 10 to about 30 ml, about 15 to about 25 ml, or about 20 ml, per gram of Telatinib. In step (ii), the methane sulfonic acid is preferably used in an amount of about 0.9 to about 1.1, or about 1, mole equivalent relative to Telatinib. Preferably, the methane sulfonic acid is added to the suspension of Telatinib in the ether.

According to any aspect or embodiment the process for preparing Telatinib mono mesylate Form TM2 may comprise: combining a suspension of Telatinib, optionally Telatinib form T2, in methyl tert-butyl ether, with methanesulfonic acid, at room temperature to form a mixture; optionally stirring the mixture; optionally isolating the solid from the mixture, preferably by filtration; and optionally drying the solid. Preferably, the process for preparing Telatinib mono mesylate Form TM2 comprises: combining a suspension of Telatinib, optionally Telatinib form T2, in methyl tert-butyl ether, with methanesulfonic acid, at room temperature to form a mixture; stirring the mixture; optionally isolating the solid from the mixture, preferably by filtration; and optionally drying the solid. According to any embodiment of this process.

The combining temperature and time, amount of the ether, amount of methane sulfonic acid, order of addition, mixture stirring time and temperature, isolation, and drying steps in this embodiment can be in accordance with any of the above-described embodiments.

Preferably, in any embodiment of the process for preparing Form TM2 as described herein, the process is conducted under ambient relative humidity conditions. Preferably, the isolating step is carried out by filtration, most preferably wherein the filtration is carried out under ambient relative humidity conditions, for example at a temperature of: about 22° C. to about 27° C., or about 25° C., and a relative humidity of about 40% to about 60%, or a relative humidity of about 45% to about 50%.

Preferably, in any embodiment of the process for preparing Form TM2, the isolated solid is dried, more preferably at a temperature of about 40° C. to about 70° C., about 50° C. to about 65° C., about 55° C. to about 65° C., or about 60° C. The drying time may be for a period of about 20 minutes to about for 180 minutes, about 30 minutes to about 100 minutes, about 40 minutes to about 80 minutes, or about 60 minutes.

The process for preparing Telatinib mono mesylate Form TM2 as described in any aspect or any embodiment of the present disclosure may further include a step of combining the Telatinib mono mesylate Form TM2 with at least one pharmaceutically acceptable excipient to form a pharmaceutical composition of pharmaceutical formulation.

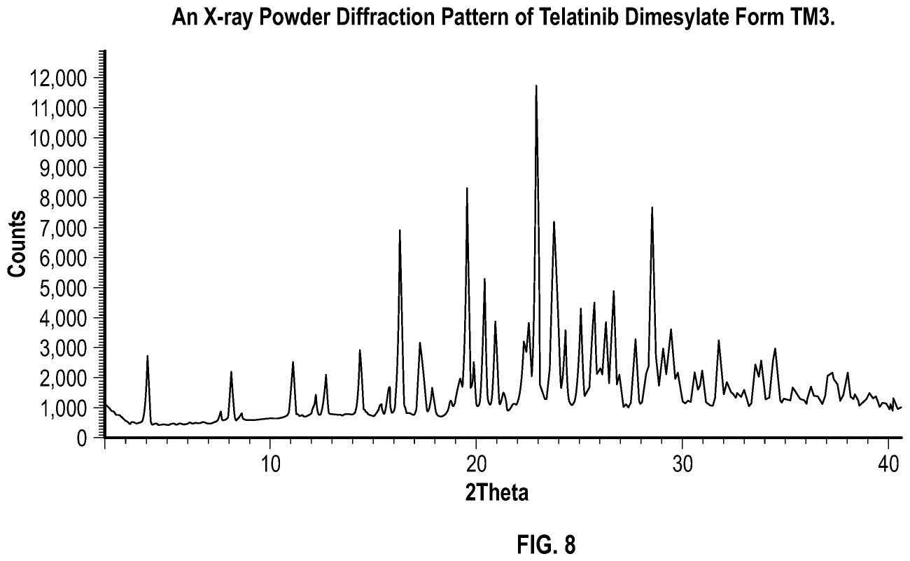

The present disclosure further includes a crystalline form of Telatinib dimesylate designated as Form TM3. Telatinib dimesylate Form TM3 can be characterized by data selected from one or more of the following: an XRPD pattern having peaks at 11.1, 12.2, 14.4, 25.1 and 27.8 degrees 2-theta±0.2 degrees 2-theta; an XRPD pattern as depicted in FIG. 8; or combinations of these data.

Telatinib dimesylate Form TM3 may be further characterized by an XRPD pattern having peaks at 11.1, 12.2, 14.4, 25.1 and 27.8 degrees 2-theta±0.2 degrees 2-theta, and also having one, two, three, four or five additional peaks at 17.3, 17.9, 22.6, 29.5 and 34.6 degrees 2-theta±0.2 degrees 2-theta.

Telatinib dimesylate Form TM3 may alternatively be characterized by an XRPD pattern having peaks at 11.1, 12.2, 14.4, 17.3, 17.9, 22.6, 25.1, 27.8, 29.5 and 34.6 degrees 2-theta±0.2 degrees 2-theta.

Telatinib dimesylate Form TM3 may be characterized by each of the above characteristics alone or by all possible combinations, e.g., by an XRPD pattern having peaks at 11.1, 12.2, 14.4, 25.1 and 27.8 degrees 2-theta±0.2 degrees 2-theta and/or an XRPD pattern as depicted in FIG. 8.

According to any aspect or embodiment of the present disclosure, Telatinib dimesylate Form TM3 is polymorphically pure. Optionally, Telatinib dimesylate Form TM3 may contain: about 20% or less, about 10% or less, about 5% or less, about 2% or less, about 1% or less, about 0.5% or less, of any other solid state forms of the subject compound as measured, for example, by XRPD.

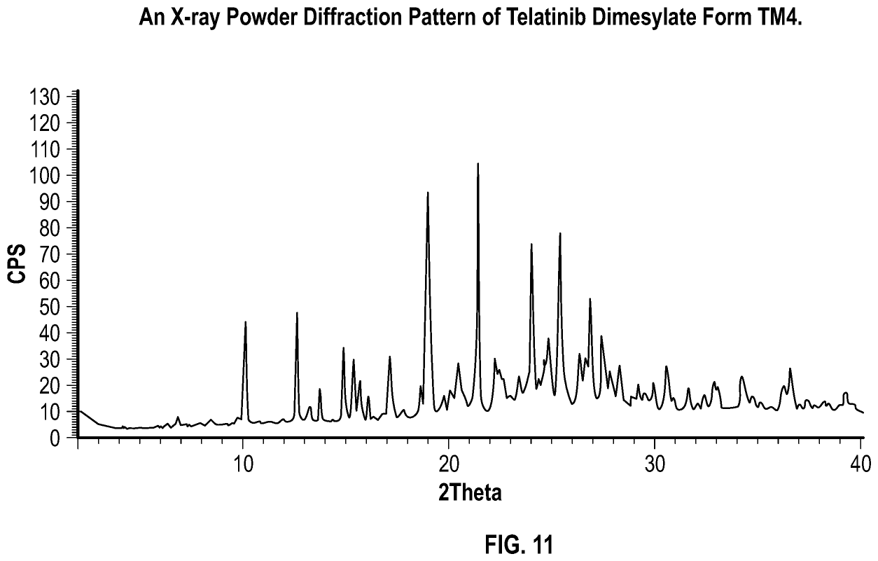

The present disclosure further includes a crystalline form of Telatinib dimesylate designated as Form TM4. Telatinib dimesylate Form TM4 can be characterized by data selected from one or more of the following: an XRPD pattern having peaks at 13.7, 14.9, 24.0, 25.4 and 34.2 degrees 2-theta±0.2 degrees 2-theta; an XRPD pattern as depicted in FIG. 11; or combinations of these data.

Telatinib dimesylate Form TM4 may be further characterized by an XRPD pattern having peaks at 13.7, 14.9, 24.0, 25.4 and 34.2 degrees 2-theta±0.2 degrees 2-theta, and also having one, two, three, four or five additional peaks at 6.9, 13.2, 18.6, 21.4, 26.4 and 27.5 degrees 2-theta±0.2 degrees 2-theta. Telatinib dimesylate Form TM4 may be alternatively be characterized by an XRPD pattern having peaks at 13.7, 14.9, 24.0, 25.4 and 34.2 degrees 2-theta±0.2 degrees 2-theta, and also having one, two, three, four, five, or six, additional peaks at 6.9, 13.2, 18.6, 21.4, 26.4 and 27.5 degrees 2-theta±0.2 degrees 2-theta.

Telatinib dimesylate Form TM4 may alternatively be characterized by an XRPD pattern having peaks at 6.9, 13.2, 13.7, 14.9, 18.6, 21.4, 24.0, 25.4, 26.4, 27.5 and 34.2 degrees 2-theta±0.2 degrees 2-theta.

Telatinib dimesylate Form TM4 may be characterized by each of the above characteristics alone or by all possible combinations, e.g., by an XRPD pattern having peaks at 13.7, 14.9, 24.0, 25.4 and 34.2 degrees 2-theta±0.2 degrees 2-theta and/or an XRPD pattern as depicted in FIG. 11.

According to any aspect or embodiment of the present disclosure, Telatinib dimesylate Form TM4 is polymorphically pure. Optionally, Telatinib dimesylate Form TM4 may contain: about 20% or less, about 10% or less, about 5% or less, about 2% or less, about 1% or less, about 0.5% or less, of any other solid state forms of the subject compound as measured, for example, by XRPD.

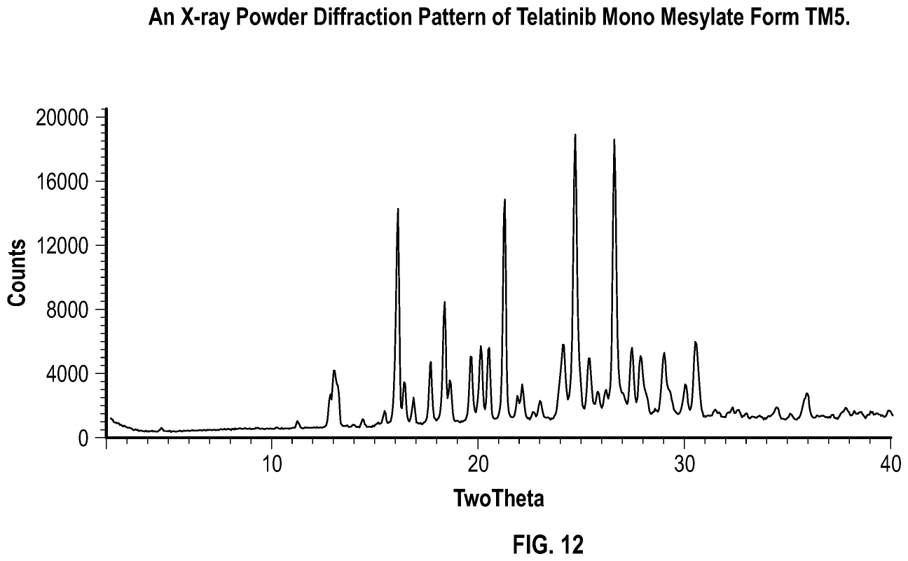

The present disclosure further includes a crystalline form of Telatinib mono mesylate designated as Form TM5. Telatinib mono mesylate Form TM5 can be characterized by data selected from one or more of the following: an XRPD pattern having peaks at 18.3, 18.6, 20.1, 27.8 and 30.0 degrees 2-theta±0.2 degrees 2-theta; an XRPD pattern as depicted in FIG. 12; or combinations of these data.

Telatinib mono mesylate Form TM5 may be further characterized by an XRPD pattern having peaks at 18.3, 18.6, 20.1, 27.8 and 30.0 degrees 2-theta±0.2 degrees 2-theta, and also having one, two, three, four, or five additional peaks at 19.6, 21.9, 25.3 and 35.9 degrees 2-theta±0.2 degrees 2-theta. Telatinib mono mesylate Form TM5 may be further characterized by an XRPD pattern having peaks at 18.3, 18.6, 20.1, 27.8 and 30.0 degrees 2-theta±0.2 degrees 2-theta, and also having one, two, three, or four additional peaks at 19.6, 21.9, 25.3 and 35.9 degrees 2-theta±0.2 degrees 2-theta.

Telatinib mono mesylate Form TM5 may alternatively be characterized by an XRPD pattern having peaks at 18.3, 18.6, 19.6, 20.1, 21.9, 25.3, 27.8, 30.0 and 35.9 degrees 2-theta 0.2 degrees 2-theta.

Telatinib mono mesylate Form TM5 may be characterized by each of the above characteristics alone or by all possible combinations, e.g., by an XRPD pattern having peaks at 18.3, 18.6, 20.1, 27.8 and 30.0 degrees 2-theta±0.2 degrees 2-theta and/or an XRPD pattern as depicted in FIG. 12.

Telatinib mono mesylate Form TM5 may be anhydrous form.

According to any aspect or embodiment of the present disclosure, Telatinib mono mesylate Form TM5 is isolated.

According to any aspect or embodiment of the present disclosure, Telatinib mono mesylate Form TM5 is polymorphically pure. Optionally, Telatinib mono mesylate Form TM5 may contain: about 20% or less, about 10% or less, about 5% or less, about 2% or less, about 1% or less, about 0.5% or less, of any other solid state forms of the subject compound as measured, for example, by XRPD.

As discussed above, depending on which other solid state form it is compared with, Telatinib mono mesylate Form TM5 according to any aspect or embodiment of the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

The present disclosure also provides the use of the solid state forms of Telatinib and Telatinib mesylate of the present disclosure for preparing different solid state forms of Telatinib and Telatinib mesylate.

The present disclosure further encompasses processes for preparing solid state forms of Telatinib and Telatinib mesylate of the present disclosure. The disclosure further includes processes for preparing different solid state forms of Telatinib and Telatinib mesylate. The process includes preparing at least one of the solid state forms Telatinib and Telatinib mesylate of the present disclosure, and converting it to different solid state forms of Telatinib and Telatinib mesylate.

In another embodiment the present disclosure encompasses the above solid state forms Telatinib and Telatinib mesylate for use in the preparation of pharmaceutical compositions and/or formulations, in some embodiments for the treatment of cancer, or in some embodiments for the treatment of gastric cancer, gastroesophageal junction cancer, hepatocellular carcinoma, advanced solid tumors, and pseudomyogenic hemangioendothelioma, and preferably gastric cancer.

In another embodiment the present disclosure encompasses the use of the above described solid state forms of Telatinib and Telatinib mesylate for the preparation of pharmaceutical compositions and/or formulations.

The present disclosure further provides pharmaceutical compositions including solid state forms of Telatinib and Telatinib mesylate of the present disclosure.

In yet another embodiment, the present disclosure encompasses pharmaceutical formulations including solid state forms of Telatinib and Telatinib mesylate of the present disclosure, and at least one pharmaceutically acceptable excipient.

Pharmaceutical formulations of the present disclosure contain any one or a combination of the solid state forms of Telatinib and Telatinib mesylate of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, the active ingredient and any other solid excipients may be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

In embodiments, a pharmaceutical formulation of Telatinib and Telatinib mesylate is formulated for administration to a mammal, such as a human. Telatinib and Telatinib mesylate can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The present disclosure encompasses a process to prepare said formulations of solid state forms of Telatinib and Telatinib mesylate thereof by combining the solid state forms of Telatinib and Telatinib mesylate according to the present disclosure and at least one pharmaceutically acceptable excipient.

Solid state forms of Telatinib and Telatinib mesylate as defined herein, as well as the pharmaceutical compositions or formulations of Telatinib and Telatinib mesylate can be used as medicaments, in some embodiments for the treatment of cancer, or in some embodiments for the treatment of gastric cancer, gastroesophageal junction cancer, hepatocellular carcinoma, advanced solid tumors, and pseudomyogenic hemangioendothelioma, and preferably gastric cancer.

The present disclosure also provides a method of treating cancer, or a method of treating gastric cancer, gastroesophageal junction cancer, hepatocellular carcinoma, advanced solid tumors, and pseudomyogenic hemangioendothelioma, and more particularly gastric cancer, by administering a therapeutically effective amount of any one or a combination of the solid state forms of Telatinib and Telatinib mesylate prepared according to the present disclosure, or at least one of the above pharmaceutical compositions or formulations, to a subject suffering from cancer, or gastric cancer, gastroesophageal junction cancer, hepatocellular carcinoma, advanced solid tumors, and pseudomyogenic hemangioendothelioma, and particularly to a subject suffering from gastric cancer, or otherwise in need of the treatment.

The present disclosure also provides the use of solid state forms of Telatinib and Telatinib mesylate, or at least one of the above pharmaceutical compositions or formulations for the manufacture of a medicament for treating cancer or for the manufacture of a medicament for treating gastric cancer, gastroesophageal junction cancer, hepatocellular carcinoma, advanced solid tumors, and pseudomyogenic hemangioendothelioma, and particularly gastric cancer.

Having described the solid state forms of Telatinib and Telatinib mesylate with reference to certain exemplary embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

### Powder X-Ray Diffraction Pattern (“XRPD”) Method:

The X-ray powder diffraction pattern was measured with an X-Ray powder diffractometer Bruker D8 Advance; CuKα radiation (λ=1.5418 Å); Lynx eye detector; laboratory temperature 22-25° C.; PMMA specimen holder ring with silicon low background. Prior to analysis, the samples were gently ground by means of mortar and pestle in order to obtain a fine powder. The ground sample was adjusted into a cavity of the sample holder and the surface of the sample was smoothed by means of a cover glass. Scanning parameters: angle range: 2-40 deg., scan mode: continuous; step size: 0.05 degrees; time per step: 0.5 s; sample spin: 30 rpm.

### EXAMPLES

### Preparation of Starting Material

Telatinib and Telatinib mesylate can be prepared according to any procedure known in the art, for example the procedure for Telatinib is described in U.S. Pat. No. 6,689,883 and the procedure for Telatinib mesylate Mod. I is described in International Publication No. WO 2008/188495.

### Example 1: Preparation of Telatinib Form T1

Telatinib mesylate Mod. I (0.5 grams) was suspended in 5 mL of 50:50 v/v Isopropyl alcohol: Water mixture at 25° C. The suspension was stirred at 25° C. for about 1 day. The obtained solid was isolated by filtration and analyzed by XRPD to contain Telatinib Form T1 (FIG. 1).

### Example 2: Preparation of Telatinib Form T1

Telatinib mesylate Mod. I (0.02 grams) was suspended in 1 mL of 50:50 v/v 2-ethoxyethanol: Water mixture at 25° C. The suspension was stirred at 25° C. for about 3 days. The obtained solid was isolated by filtration and analyzed by XRPD to contain Telatinib Form T1.

### Example 3: Preparation of Telatinib Form T2

Telatinib Monohydrate Form T1 (0.1 grams) was suspended in 3 mL of Isopropyl alcohol at 25° C. The suspension was stirred at 25° C. for about 1 day. The obtained solid was isolated by filtration and analyzed by XRPD to contain Telatinib Form T2.

### Example 4: Preparation of Telatinib Form T3

Telatinib mesylate Mod. I (0.05 grams) was dissolved in 0.2 mL of N,N-Dimethylformamide at 25° C. The clear solution was cooled immediately to −70° C. (dry ice/Acetone) and maintained at −70° C. for about 1 hour and the clear solution placed in the room temperature for slow solvent evaporation. After 24 hours, solids were observed and the obtained solid was filtered and dried under vacuum at 25° C. for about 10-15 minutes and analyzed by XRPD and to contain Telatinib Form T3.

### Example 5: Preparation of Telatinib Form T4

Telatinib mesylate (0.02 grams) was dissolved in 0.5 mL of Ethylene Glycol at 25° C. The clear solution was covered with aluminum foil with pinhole and subjected to solvent evaporation at 25° C. for 7 days. The obtained solid was filtered, dried under vacuum at 25° C. for about 10-15 minutes and analyzed by XRPD to contain Telatinib Form T4.

### Example 6: Preparation of Telatinib Dimesylate Form TM1

Telatinib Form T1, prepared following examples 1 or 2 (0.05 grams) was suspended in 1 mL of ethyl acetate for about 10 minutes at 25° C. under stirring. Methanesulfonic acid (0.01 mL, 1 mole equivalent) was added and the suspension was kept for 2 hours at 25° C. under stirring. The obtained solid was isolated by filtration and dried under vacuum at 25° C. for 30 minutes and analyzed by XRD to contain Telatinib dimesylate Form TM1.

### Example 7: Preparation of Telatinib Mono Mesylate Form TM2

Telatinib Form T2, prepared following example 3 (0.05 grams) was suspended in 1 mL of methyl tert-butyl ether for about 30 minutes at 25° C. under stirring. Methanesulfonic acid (0.01 mL, 1 mole equivalent) was added and the suspension was kept for 2 hours at 25° C. under stirring. The obtained solid was isolated by filtration and dried under vacuum at 60° C. for 60 minutes and analyzed by XRD to contain Telatinib mono mesylate Form TM2.

### Example 8: Preparation of Telatinib Form T5

Telatinib mesylate Mod. I or Telatinib Mesylate Form TM2 (0.5 grams) was suspended in 5 mL of 50:50 v/v 1,4-Dioxane:Water mixture at 25° C. for about 1 day under stirring. The obtained solid was isolated by filtration (drying under suction for 15-20 minutes at RT) and analyzed by XRD to obtain Telatinib Form T5 (FIG. 9).

### Example 9: Preparation of Telatinib Form T6

Telatinib form T5 (0.9 grams) was suspended in 10 mL of methyl tert-butyl ethane (MTBE) at 25° C. for about 3 days under stirring. The obtained solid was isolated by filtration and analyzed by XRD to obtain Telatinib Form T6 (FIG. 10).

### Example 10: Preparation of Telatinib Dimesylate Form TM3

Telatinib Form T2 (0.03 grams) was suspended in 1 mL of Isopropyl Acetate at 25° C. for about 30 minutes under stirring. Methanesulfonic acid (0.01 mL, 2 mole equivalent) was added, stirred for 2 hours and isolated by filtration to obtain Telatinib dimesylate Form TM3 (FIG. 8).

### Example 11: Preparation of Telatinib Dimesylate Form TM4

Telatinib Form T1 (0.05 grams) was suspended in 1 mL of methyl Isopropyl ketone (MIPK) for about 30 minutes, at 25° C. under stirring. Methanesulfonic acid (0.01 mL, 1 mole equivalent) was added and kept for 2 hours. The obtained solid was isolated by filtration and analyzed by XRD to contain Telatinib dimesylate Form TM4 (FIG. 11).

### Example 12: Preparation of Telatinib Dimesylate Form TM1

Telatinib mesylate Mod. I (0.1 grams) was suspended in 0.5 mL of 10% (v/v) Methanesulfonic acid in ethanol for 18 hours at 25° C., under stirring. The obtained solid was isolated by filtration and dried under vacuum for about 10-15 minutes to obtained Telatinib dimesylate Form TM1.

### Example 13. Preparation of Telatinib Mono Mesylate Form TM2

Telatinib mono mesylate (3.0 grams) was dissolved in 40 mL Dimethylacetamide (DMAc) at 60° C. (clear solution was observed after 15-20 minutes), filtered through 0.45 micron filter at 25° C. to remove any undissolved particles (this solution was defined stock solution and kept aside). About 5% seeds of TM2 was added to 300 ml cold n-Butylacetate and kept for about 5 minutes at 0-5° C. with stirring (250 rpm). Then, the previous prepared stock solution was added slowly drop wise for about 10 min (immediate precipitation was observed). The reaction mixture was maintained for 10-15 minutes, filtered, dried under vacuum for 15-30 minutes and additional drying at 50-60° C. for 1 hour to obtain Telatinib mono mesylate form TM2.

### Example 14. Preparation of Telatinib Mono Mesylate Form TM5

Telatinib mono mesylate form TM2 (0.1 grams) was packed in low density polyethylene bag which was twist and tied with cable tie and this was kept in triple laminated aluminum bag which was heat sealed, and incubated in 25° C./60% RH and 40° C./75% RH conditions for 6 months. The solid was measured after 6M by XRD to obtain Telatinib mono mesylate designated Form TM5.

1. A crystalline form of Telatinib mono mesylate, designated as Form TM2, which is characterized by an XRPD pattern having peaks at 16.0, 16.5, 18.0, 22.6 and 24.9 degrees 2-theta±0.2 degrees 2-theta.

2. A crystalline form according to claim 1, which is further characterized by an XRPD pattern having an additional peak at 19.0 degrees 2-theta±0.2 degrees 2-theta.

3. A crystalline form according to claim 1, which is further characterized by an XRPD pattern having an absence of a peak at 7.9 degrees 2-theta±0.2 degrees 2-theta.

4. A crystalline form according to claim 1, which is further characterized by having one, two, three or four additional peaks at 4.5, 12.8, 21.2 and 26.8 degrees 2-theta±0.2 degrees 2-theta.

5. A crystalline form according to claim 1, which is characterized by an XRPD pattern having peaks at 4.5, 12.8, 16.0, 16.5, 18.0, 21.2, 22.6, 24.9 and 26.8 degrees 2-theta±0.2 degrees 2-theta.

6. A crystalline form according to claim 5, which is characterized by an XRPD pattern having peaks at 4.5, 12.8, 16.0, 16.5, 18.0, 19.0, 21.2, 22.6, 24.9 and 26.8 degrees 2-theta±0.2 degrees 2-theta and the absence of peak at 7.9 degrees 2-theta±0.2 degrees 2-theta; or an XRPD pattern substantially as depicted in FIG. 7.

7. A crystalline form of Telatinib mono mesylate, designated as Form TM5, which is characterized by: an XRPD pattern having peaks at 18.3, 18.6, 20.1, 27.8 and 30.0 degrees 2-theta±0.2 degrees 2-theta.

8. A crystalline form according to claim 7, which is further characterized by an XRPD pattern having one, two, three, or four additional peaks at 19.6, 21.9, 25.3 and 35.9 degrees 2-theta±0.2 degrees 2-theta.

9. A crystalline form according to claim 7, which is characterized by an XRPD pattern having peaks at 18.3, 18.6, 19.6, 20.1, 21.9, 25.3, 27.8, 30.0 and 35.9 degrees 2-theta±0.2 degrees 2-theta; or an XRPD pattern substantially as depicted in FIG. 12.

10. A crystalline form according to claim 1, which is isolated.

11. A crystalline form according to claim 1, which contains no more than about 20% of any other crystalline forms of Telatinib mono mesylate.

12. A crystalline form according to claim 1, which contains no more than about 20% of amorphous Telatinib mono mesylate.

13. A pharmaceutical composition comprising a crystalline form according to claim 1.

14. (canceled)

15. A pharmaceutical formulation comprising a crystalline form according to claim 1 with at least one pharmaceutically acceptable excipient.

16. A process for preparing a pharmaceutical formulation comprising combining a crystalline form according to claim 1 with at least one pharmaceutically acceptable excipient.

17. A medicament comprising the crystalline form according to claim 1.

18. (canceled)

19. A method of treating cancer, optionally gastric cancer, gastroesophageal junction cancer, hepatocellular carcinoma, advanced solid tumor, or pseudomyogenic hemangioendothelioma, comprising administering a therapeutically effective amount of a crystalline form according to claim 1 to a subject in need of the treatment.

20. (canceled)

21. (canceled)

22. A crystalline form according to claim 7, which is isolated.

23. A crystalline form according to claim 7 which contains no more than about 20% of any other crystalline forms of Telatinib mono mesylate.

24. A crystalline form according to claim 7, which contains no more than about 20% of amorphous Telatinib mono mesylate.

25. A pharmaceutical formulation comprising a crystalline form according to claim 7 with at least one pharmaceutically acceptable excipient.

26. A process for preparing a pharmaceutical formulation comprising combining a crystalline form according to claim 7 with at least one pharmaceutically acceptable excipient.

27. A method of treating cancer, optionally gastric cancer, gastroesophageal junction cancer, hepatocellular carcinoma, advanced solid tumor, or pseudomyogenic hemangioendothelioma, comprising administering a therapeutically effective amount of a crystalline form according to claim 7 to a subject in need of the treatment.
